(19) United States
(12) Patent Application Publication
(10) Pub. No.: US 2026/0144487 A1
(43) Pub. Date: May 28, 2026

(54) BIOMETRIC DATA COLLECTION SYSTEM FOR SMALL ANIMALS UTILIZING GLOVES WITH MULTIPLE ELECTRODES

(71) Applicant: Mouse Specifics, Inc., Framingham, MA (US)

(72) Inventors: Thomas G. Hampton, Framingham, MA (US); Lyla Hampton, Framingham, MA (US); Melissa Lewis, Waltham, MA (US)

(21) Appl. No.: 18/962,096

(22) Filed: Nov. 27, 2024

(51) Int. Cl. A61B 5/00; A61B 5/282

(52) U.S. Cl. CPC A61B 5/6806; A61B 5/282; A61B 5/6815; A61B 2503/40; A61B 2503/42

(57) ABSTRACT

A biometric data collecting system for small animals includes a glove with specific electrode placements for capturing biometric data. The glove features a first electrode on the index finger and thumb portions, and a second electrode on the ring finger, little finger, and palm portions below the ring finger and little finger. By holding a small animal in the palm portion of the glove with the anterior between the index finger and thumb (first electrode contact point) and the posterior between the ring finger, little finger, and palm below them (second electrode contact point), biometric data is measured and collected separately at each contact point. This system provides a convenient and efficient way to gather biometric data from small animals for various applications.

### FIELD OF THE INVENTION

The present invention relates to measurements of physiological characteristics in small animals, such as sugar gliders, mice, rats, hamsters, guinea pigs, and other small mammals, including small humans. More particularly, the present invention relates to a glove or pair of gloves imbued with electrically conductive sensors strategically placed to enable the wearer of the glove(s) to articulate the hand(s) and digits to detect and record biometric data such as electrocardiogram (ECG) of the subject in contact with the glove(s).

### BACKGROUND

Small animals, or more specifically, small mammals such as mice and rats, are routinely used in the research setting for studying a wide range of human disorders. Tens of millions of lab mice are used in preclinical translational studies, to determine the effects of genes, diseases, and drugs in convenient mammalian models of human health challenges. Moreover, there are about 1 million people in the US that keep mice and hamsters as pets. There is a need to monitor the health and well-being of such small mammals for research purposes or to ensure their good health. For humans, the health of the heart is routinely checked, either by recording a blood pressure, checking the pulse, or recording an electrocardiogram. In small mammals, however, this is not routine, as small animals such as mice can move very fast, try to escape measurement, and attempt to bite whoever is handling the animal. Therefore, small mammals are often anesthetized or sedated in order to gauge heart health. However, it is well known that the anesthetic or sedation can profoundly affect the true metrics of heart function and health. Monitoring of the heart rate and electrocardiogram in some animals is often performed by inserting pin electrodes into the sedated subject's appendages, affixing the animal's forelimbs and hind limbs to conductive electrodes, or surgically inserting implants. The imposition of the pin electrodes can cause pain and tissue damage, the affixing of limbs to conductive electrodes is often cumbersome, and the surgical implants can require weeks of recovery. Also, it is well known that anesthetic has a profound effect on the heart, so most often times the ECG from an awake subject is preferable. Therefore, a need exists for improved methodologies to record the ECG from awake small mammals.

There is a movement towards home cage monitoring of laboratory animals, by way of sensors build into the animals housing and use of cameras, AI, and data. Nonetheless, laboratory animals are routinely handled by an animal technician, husbandry staff, and veterinarian staff, to intimately examine the subjects, administer drugs, or to extract fluids such as blood. It is unlikely that the need or desire to physically pick up a laboratory mouse in preclinical research will go away, and it is likely that the need for veterinary staff or medical staff to pick up a small animal or small human for diagnosis and care will continue.

Yet there is also a growing awareness and desire for any manual handling of a laboratory animal to be done with the greatest care, while minimizing stress. It is widely accepted that just the very action of picking up an animal is stressful to the animal, though the data reflecting that degree of stress is not widely known or is subject to discussion. The heart rate of a mouse is from about 400 bpm when the animal is resting quietly to above 800 bpm when the animal is actively moving. Unpublished data show that the heart rate of a mouse that is picked up and held for a few moments is also ˜800 bpm, so it is difficult to conclude decidedly that the animal is in a state of stress.

Routine intraperitoneal (i.p.) administration of drugs is widely performed scores of time in millions of laboratory animals each year, extensively in the drug development and testing of new compounds for human use. It is fairly standard practice for a researcher to “scruff” the animal with one hand, typically the index finger and thumb of their hand, essentially pinching the fir and loose skin below the fur just behind the head of the animal atop of its head, and to ensnare the animal's tail below the ring finger and/or little finger of the same hand, and rotate the hand such that the splayed ventral surface of the animal is presented to the researcher. So positioned, the belly of the subject is available and somewhat pronounced to a needle held in the other hand, with which the injection of compound inside the needle to the belly of the subject is easily accomplished. Once accomplished, the animal can be returned to its habitat. Subcutaneous administration of drugs and other injections of drugs or withdrawal of biological fluids also typically require handling of the animal. Routinely, and in some places prescribed by policy if not law, the researcher is required to wear gloves, typically disposable latex gloves, when working with animals and performing such procedures.

To know the health of a laboratory animal or to examine the potential effect of a new drug on the heart, it is routine for veterinary staff and researchers to monitor the heart of the animal. This might be accomplished in the anesthetized animals, or by way of surgical implantation of radio transmitters, or by way of technology developed by this inventor and in use today.

U.S. Pat. No. 6,445,941 previously described an invention whereby the small animal stands on an array of conductive electrodes in order to record the ECG of the subject, possibly before the injection of the drug described in the above paragraph, or afterward. U.S. Pat. No. 8,649,086 described an invention having two points of electrical contact crossing the subject's heart. The data capture is greatly improved when the subject's range of motion is limited. U.S. Pat. No. 10,959,399 describes a disposable habitat for obtaining the electrical signals of the subject through its feet and possibly tail. U.S. Pat. No. 7,065,396 describes using the ear of a mammal as a means for obtaining the ECG signal but does not make obvious the method and device for accomplishing such herein described.

### SUMMARY

Therefore, a need exists for new methods and devices to determine the biometric data, such as heart rate and ECG, of a laboratory animals or small companion animals as they are being held by the hand of a veterinary caretaker medical staff or the animal's guardian.

Embodiments of the present invention incorporate the structure, height, length, and material of the glove and conductive ECG recording material inherent or adhered to the glove itself to make the ECG recording glove affordable to manufacture and sell at a reasonable price. Latex, for example, of which rubber gloves are ubiquitously available, serves this purpose. Imbued into the material, either by process of the glove material, or after the glove per se is manufactured, is electrically conductive material designed and configured for the purpose of recording the electrical activity of the heart.

Typically, in humans and in animals, two points of electrical contact are required to measure the small bioelectrical potential across the heart, typically one on one side of the heart, and another on an opposite side of the heart. With two points of contact connected electrically to a bio amplifier and voltage detection device, the size and frequency of the potential can be registered and record, the so-called electrocardiogram or ECG.

Because the “scruffing” procedure is typically performed with the thumb and index finger, electrically conductive material is strategically and sufficiently placed around the tips of the fingers of the gloves and nearly circumferentially at either or both of these locations. Means to electrically connect the thumb and index finger are employed such that either finger alone or both fingers together constitute one of the poles with which to sense the heart's electrical activity through the scruffed neck or parts nearby such as the ears.

Because the tail of a laboratory mouse or rat is typically ensnared by the ring finger and/or the little finger during, for example, the i.p. administration of drugs, electrically conductive material is strategically and sufficiently placed around the tips of these fingers and along the length the ventral surfaces of the fingers extending to an including the palm of the hand, at least towards the right side of the right palm, or the left side of the left palm. This strategic placement and pattern of the conductive material increases the likelihood of good electrical contact between a distal point and a proximal point on the subject's body to render a detectable ECG signal in concert with the point of contact made with the digit(s) performing the scruffing.

Here, with the present disclosure, we describe a glove in which the electrically conductive electrodes for recording the biometric data are built into the glove itself. When the glove is attached to the data-capturing ECG bio-amplifier, the electrical signal of the heart of the subject being held by the researcher in the gloved hand(s) reflects the ECG and heart rate of the animal as it is being held. In this regard, the researcher may (a) know the heart rate of said subject at this time of being held by the researcher; (b) wait to administer the compound until a particular heart rate is registered; (c) see immediate and short-term effects of the compound on the heart function of the animal.

In accordance with embodiments of the present invention, a biometric data collecting system for small animals is provided. The system includes a glove, a first electrode, and a second electrode. The glove has a thumb, index finger, middle finger, ring finger, little finger, and palm portions. The first electrode involves a conductive material disposed on the index finger portion and the thumb portion of the glove. The second electrode is electrically isolated from the first electrode and involves a conductive material disposed on the ring finger portion, little finger portion, and the palm portion below the ring finger portion and the little finger portion of the glove. When a small animal is held by a wearer of the glove generally in the palm portion, an anterior of the animal is held between the index finger portion and thumb portion creating a first point of contact for the first electrode on the animal, and a posterior of the animal is held between the ring finger portion and/or little finger portion and the portion of the palm below the ring finger portion and little finger portion creating a second point of contact for the second electrode on the animal separate from the first point of contact. Biometric data is measured and collected using the first point of contact for the first electrode on the animal and the second point of contact for the second electrode on the animal separate from the first point of contact.

In accordance with aspects of the present invention, the biometric data is electrocardiogram (EKG/ECG) data.

In accordance with aspects of the present invention, the system further includes connectors that connect the first electrode and second electrode to biometric data collection devices. In some such aspects, the connectors are magnetic connectors.

In accordance with aspects of the present invention, the glove is configured in a left-hand orientation or a right-hand orientation.

In accordance with aspects of the present invention, wherein the electrically conductive material comprises a silver film, or a silver chloride film. In other aspects, the electrically conductive material comprises a conductive ink or paint.

In accordance with aspects of the present invention, the glove comprises a latex or polymer glove. In other aspects, the glove comprises a leather or cloth glove.

In accordance with aspects of the present invention, the system further includes a compressible material disposed between the palm of the glove and the conductive material of the second electrode on the portion of the palm below the ring and little finger.

In accordance with aspects of the present invention, the system further includes a heating element disposed between the palm of the glove and the conductive material of the second electrode on the portion of the palm below the ring and little finger.

In accordance with aspects of the present invention, the system further includes a force sensor disposed between the palm of the glove and the conductive material of the second electrode on the portion of the palm below the ring and little finger.

In accordance with aspects of the present invention, the system further includes extensions of the first electrode disposed on the index finger and thumb configured to contact ears of the animal when the anterior of the animal is held between the index finger and thumb. In certain aspects, the extensions comprise a compressible conductive material. In some such aspects, the compressible conductive material comprises a sponge.

In accordance with aspects of the present invention, the system further includes a conductive medium applied to the first electrode disposed on the index finger and thumb. In some such aspects, the conductive medium is contained in vesicles disposed in fingertips of the glove where a pressure of holding the anterior of the animal causes the conductive medium to be expelled from the vesicles and applied to the first electrode disposed on the index finger and thumb. In further such aspects, the conductive medium is expelled from the vesicles by pressure applied by an adjacent finger on the vesicle.

In accordance with aspects of the present invention, the glove is scented.

In accordance with aspects of the present invention, the system further includes a second glove, a third electrode, and a fourth electrode. The second glove has a thumb, index finger, middle finger, ring finger, little finger, and palm portions. The third electrode involves a conductive material disposed on the index finger portion and thumb portion of the second glove. The fourth electrode is independent of the third electrode and involves a conductive material disposed on the ring finger portion, little finger portion, and area of the palm portion below the ring finger portion and little finger portion of the second glove. The third electrode establishes a third point of contact and the fourth electrode establishes a fourth point of contact. Biometric data can be measured using combination of any of: the first point, second point, third point, and fourth point of contact on the small animal.

In accordance with embodiments of the present invention, a method of using a biometric data collecting system for small animals is provided. The method involves providing a biometric data collecting system for small animals as disclosed herein; wearing the biometric data collecting system on a hand of a user; holding a small animal generally in the palm portion with an anterior of the animal held between the index finger portion and thumb portion creating a first point of contact for the first electrode on the animal, and a posterior of the animal held between the ring finger portion and/or little finger portion and the portion of the palm below the ring finger portion and little finger portion creating a second point of contact for the second electrode on the animal separate from the first point of contact; and measuring and collecting biometric data using the first point of contact for the first electrode on the animal and the second point of contact for the second electrode on the animal separate from the first point of contact.

In accordance with aspects of the present invention, the anterior of the animal includes the loose skin on a neck of the animal.

In accordance with aspects of the present invention, the posterior of the animal includes the tail of the animal.

### BRIEF DESCRIPTION OF THE FIGURES

These and other characteristics of the present invention will be more fully understood by reference to the following detailed description in conjunction with the attached drawings, in which:

FIG. 1 depicts a biometric data collecting system for small animals in accordance with embodiments of the present invention;

FIG. 2 depicts the use of biometric data collecting system for small animals in accordance with embodiments of the present invention;

FIG. 3 depicts another view of the biometric data collecting system for small animals in use in accordance with embodiments of the present invention;

FIG. 4 depicts another way of using the biometric data collecting system for small animals in use in accordance with embodiments of the present invention;

FIG. 5 depicts another embodiment of the biometric data collecting system for small animals featuring two gloves;

FIG. 6 depicts the use of biometric data collecting system for small animals featuring two gloves in accordance with embodiments of the present invention; and

FIG. 7 depicts an example of a vesicle disposed on a fingertip that can be used to apply a conductive medium used in collecting biometric data gloves in accordance with embodiments of the present invention.

### DETAILED DESCRIPTION

An illustrative embodiment of the present invention relates to a biometric data collecting system for small animals comprising a glove in which the electrically conductive electrodes for recording the biometric data are built into the glove itself. When the glove is attached to the data capturing device, the electrical signal of the heart of the subject being held by the researcher in the gloved hand(s) reflects the electrocardiogram (EKG/ECG) and heart rate of the animal as it is being held. In this regard, the researcher may a. know the heart rate of said subject at this time of being held by the researcher; b. wait to administer the compound until a particular heart rate is registered; c. see immediate and short-term effects of the compound on the heart function of the animal.

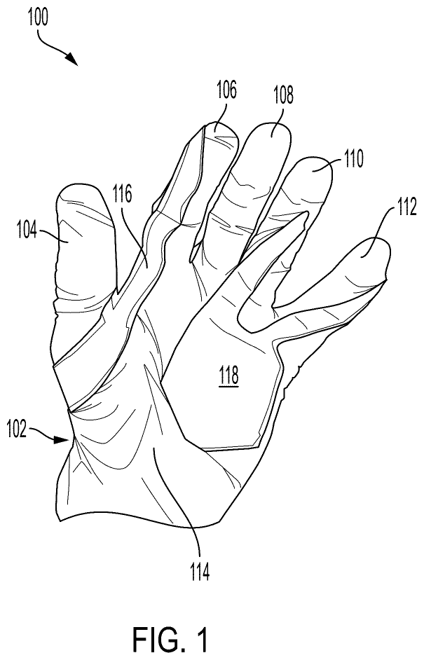

FIG. 1 through FIG. 7 wherein like parts are designated by like reference numerals throughout, illustrate an example embodiment or embodiments of a biometric data collecting system for small animals, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

FIG. 1 depicts one embodiment of a biometric data collecting system 100 for small animals. The system comprises a glove 102 comprising thumb 104, index finger 106, middle finger 108, ring finger 110, little finger 112, and palm 114 portions. The glove is designed to worn on a hand of a user. A first electrode 116 comprises a conductive material disposed on the index finger 106 portion and the thumb 104 portion of the glove 102. A second electrode 118, electrically isolated from the first electrode 116, comprises a conductive material disposed on the ring finger 110 portion, little finger 112 portion, and the palm 114 portion below the ring finger 110 portion and the little finger 112 portion of the glove 102. In this example, a conductive material has been applied down the index finger 106 and up a dorsal side of the glove 102 up to the tip of the thumb 104 to make the first electrode 116. The palm 114 and smaller digits 110, 112 also swathed with another swatch of conductive material to make the second electrode 118.

In one embodiment, the electrically conductive material is graphite paint, silver paint, or any conductive ink or paint applied to the glove 102 either by hand or by machine, creating the strategic pattern for said purpose. In another embodiment, the electrically conductive material is a silver film, or a silver chloride film that is cut in a pattern and adhered to the glove 102, creating the strategic pattern for said purpose. In still other embodiments, the entirety of the glove 102 is produced from a conductive material, with means to electrically separate the distal portion of the glove from the proximal portion of the glove, so that the strategic pattern is achieved.

In one embodiment, a latex glove or other standard polymer glove (nitrile, etc.) is prepared in order to mask certain portions of the glove prior to being dipped in or sprayed in a conductive medium. For example, in light of the above description, a strip of masking tape, say 1 cm wide by ˜25 cm long could be applied in a line extending up and down the middle finger 108 and to the cuff of the glove on the dorsal and ventral surfaces. Dipping or spraying the glove 102 with a conductive medium would cover the entire glove 102 except for the portion masked. Upon drying of the medium and removal of the tape, the resultant glove 102 has two conductive portions that serve the intended purpose as described above. Obviously, more or less intricate applications of the masking tape or masking process would result in a more intricate or strategic pattern of conductive material vs. non-conductive material.

In one embodiment, a swatch of bubble wrap, foam, or other compressible material, of thickness between 0.75 cm and 2 cm, cut to cover approximately 75% of the palm of the hand, is sandwiched between the glove and the conductive material, thereby facilitating contact of the conductive material with the tail, ensnared between the tail and the fingers.

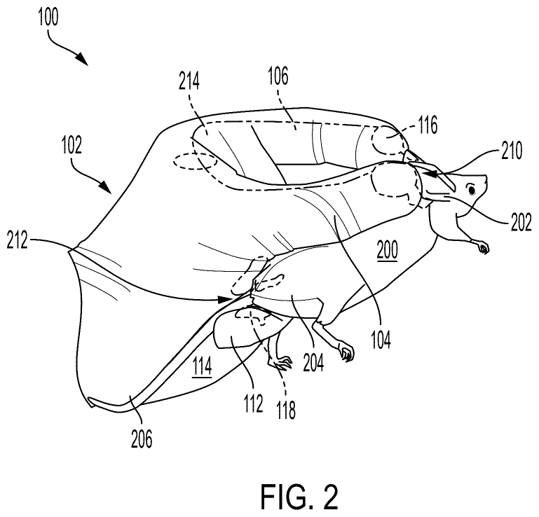

FIG. 2 depicts the of use for biometric data collecting system 100 for small animals. According to the methods and techniques presented here, the glove 102 is worn on a hand of a user. A small animal 200 can then be held by a wearer of the glove 102 generally in the palm 114 portion. An anterior 202 of the animal 200 is held between the index finger 106 portion and thumb 104 portion creating a first point of contact 210 for the first electrode 116 on the animal 200. The posterior 204 of the animal 200 is held between the ring finger 110 portion and/or little finger 112 portion and the area of the palm 114 below the ring finger 110 portion and little finger 112 portion creating a second point of contact 212 for the second electrode 118 on the animal 200 separate from the first point of contact 210. Here, for the first electrode 116, the conductive material close to the tips of the index finger 106 and/or thumb 104, the digits used to scruff an animal 200, can continue down the fingers 106, 104, for additional conduction options such as a magnetic connection point 214 for connecting to a data capture device, or along the back of the glove 102 for facile connection to a data capture recording device. For the second electrode 118, the swath of conductive material is strategically located in the palm 114, extending up the ring finger 110 and little finger 112, since the tail 206 is typically ensnared between these fingers 110, 112 and the palm 114. Moreover, the pattern(s) of conductive material in the aggregate provide opportunities for the subject in the hand to have unique points of contact with limbs and appendages on either the first point of contact 210 or second point of contact 212. The biometric data is measured and collected using the first point of contact 210 for the first electrode 116 on the animal 200 and the second point of contact 212 for the second electrode 118 on the animal 200 separate from the first point of contact 210.

In certain embodiments, connectors are included to attach a recording wire to the conductive material of the first electrode 116 on the proximal end of the glove 102, electrically conducting to the tips of the fingers 104, 106 performing the “scruffing” and to attach a recording wire to the conductive material on the distal end of the glove, electrically conducting the palm and the index finger and little finger, separately or independently in contact with the tail or buttocks region. In some such embodiments, connectors are included to make the attachment of the wires to the glove possible without the use of either hand or outside help. For example, a magnetically attractable material such as steel can be strategically located within the electrically conductive material at the proximal end and the distal end of the glove that will mate with magnets when either the proximal or distal ends of the glove are brought in close proximity to the magnets. These magnets can be either electrically conductive themselves or are responsible for shuttling the conductive wires that complete the circuit to the bio-amplifier and biopotential recording apparatus for the purpose of recording the ECG.

In one embodiment, 2 small flat pieces of magnetically attractable steel, likely 1 cm in diameter and <0.015 thick are sandwiched somewhere between the glove material and each of the two electrically conductive pathways on the surface of the gloves. These become anchoring points for each of the conductive wires that so configured with a small magnet will freely attach to the gloves at these points.

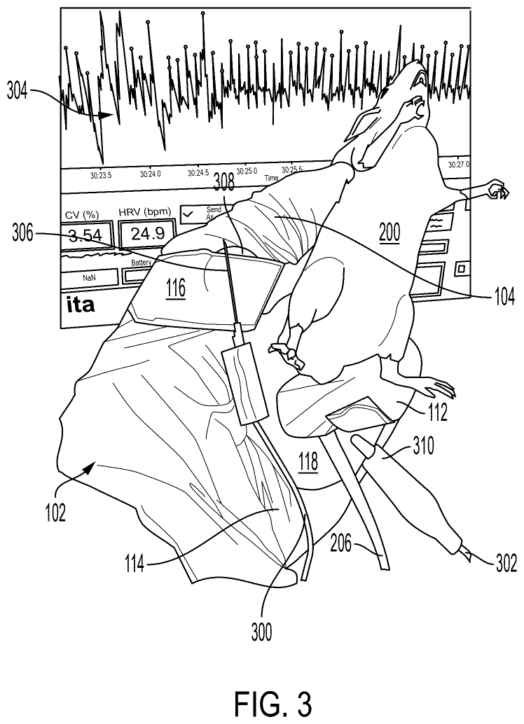

FIG. 3 depicts another view of an animal 200, being held by a user wearing the glove 102. Here, one hand is used here to scruff the animal 200, pinching the loose skin on the neck of the animal 200 with the thumb 104 and index finger 106 coated with the conductive film forming the first electrode 116, and ensnaring the tail between the little finger 112 and the palm 114, coated with the conductive film forming the second electrode 118. Shown on the left is one conducting wire 300 connected to the thumb 104, and on the palm 114 another conducting wire 302, these wires 300, 302 relay the signals to a bio-amplifier and data acquisition equipment to record and display the signal (shown on the computer screen 304). On the left, the contact is made between a steel pin 306 and a magnet 308 hidden below the conductive material of the first electrode 116 on the thumb 104. On the right, the contact is made via an alligator clip 310 clipped to the conductive material of the second electrode 118. In one embodiment, a small piece of thin steel will be hidden below the conductive material of the first 116 and second 118 electrodes, since the gloves 102 will be disposable, and the connecting wires 300, 302 will be configured with small magnets to mate to the steel shims through the conductive material, enabling the wearer of the glove to readily and easily bring the hand towards the magnets to make or break the electrical connections.

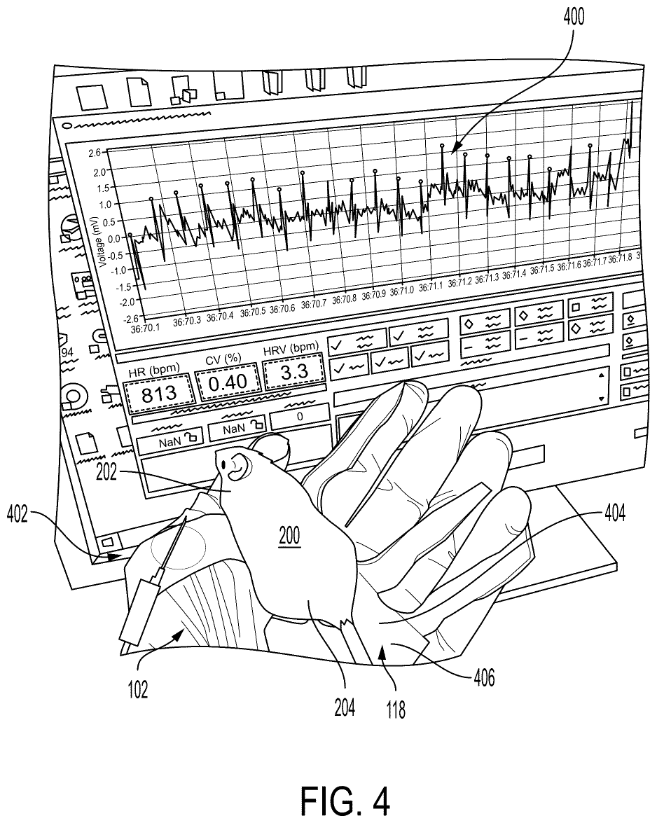

The configuration of the spacing of the first electrode 116 and second electrode 118 is not arbitrary but rather facilitates contact between a small animal's appendages, one caudally on the posterior 204 and one rostrally on the anterior 202. Here in FIG. 4 an animal 200 is resting quietly in the gloved 102 hand, with the ECG recording 400 reflected on the computer monitor 402. In certain embodiments, a heating element 404 may be disposed between the palm 114 of the glove and the conductive material of the second electrode 118 on the area of palm 114 below the ring finger 110 and little finger 112. In still other embodiments, a force sensor 406 disposed between the palm 114 of the glove 102 and the conductive material of the second electrode 118 on the area of palm 114 below the ring finger 110 and little finger 112 for detecting the subject's weight resting atop of the gloved 102 palm 114. The sensor 406 registers the animal's weight and/or breathing frequency. For example, a force transducer inside a closed region of air will register and increase in pressure inside the region, which can be computed to a weight of the object causing the change in pressure.

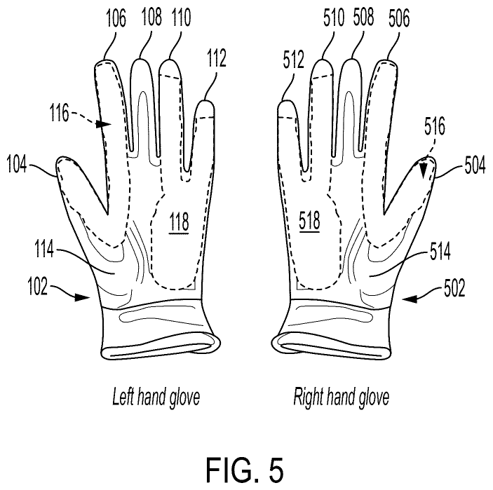

FIG. 5 depicts a biometric data collecting system 100 that includes a second glove 502. The second glove 502 comprises thumb 504, index finger 506, middle finger 508, ring finger 512, little finger 512, and palm 514 portions. A third electrode 516 comprises a conductive material disposed on the index finger 506 portion and thumb 504 portion of the second glove 502. A fourth electrode 518 independent from the third electrode 516 comprises a conductive material disposed on the ring finger 510 portion, little finger 512 portion, and area of the palm 514 portion below the ring finger 510 portion and little finger 512 portion of the second glove 502. The third electrode 516 establishes a third point of contact and the fourth electrode 518 establishes a fourth point of contact. Biometric data can be measured using combination of any of: the first point, second point, third point, and fourth point of contact on the small animal 200. Here, the darker shade represents the glove material, and the lighter shade denotes the conductive material of the first electrode 116, second electrode 118, third electrode 516, and fourth electrode 518. The placement and path of the conductive material of the first electrode 116, second electrode 118, third electrode 516, and fourth electrode 518 is strategized to optimize recording of the cardiac electrical signals of a small animal 200 in contact with the conductive material, either with just one hand, or both hands. The pattern can be accomplished via conductive inks or paints, dipping the entire glove in conductive ink but masking portions meant to be insulated, and other means for creating at least two unique conductive electrodes 116 and 118 or 516 and 518 on each glove 102, 502.

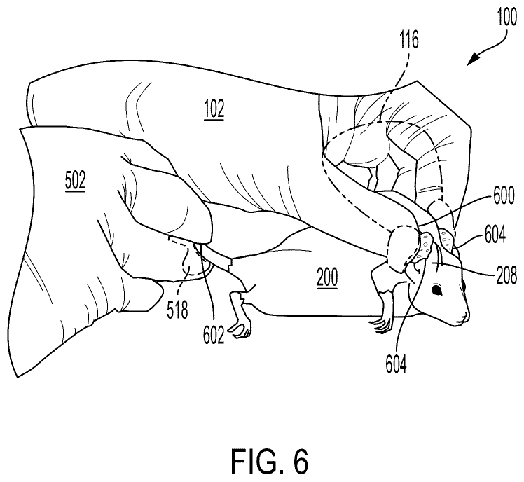

In some embodiments, it might be preferable or necessary to position the subject differently and with two hands rather than one hand. In this case, another glove 502 worn on the opposite hand, with the electrical connection made to the appropriate end of the glove 502, serves the purpose of completing the conductive pathway across the heart. An example of this is shown in FIG. 6. Here, an animal caretaker is shown carefully assessing the health of an animal 200. With the first glove 102 making a first point of contact 600 using the first electrode 116 and the second glove 502 making the second point of contact 602 using the second electrode 518. This could just as easily be an infant, with the neck being cradled in one hand, and the buttocks are of the infant being held in the other hand.

While it is possible to obtain the ECG from the scruffed portion of the neck, the inventor has noted (and also as described in patent #7, 065, 396) that the ears of small animals provide good points of contact for ECG recording. Therefore, one embodiment of this invention is the inclusion of extensions or appendages 604 to the proximal portion of the glove 102 close to the index finger 106 and thumb 104 of the conductive material of the first electrode 116, positioned so that they actively become in contact with the ears 208 of the animal 200 as the animal 200 is scruffed. In some embodiments, these extensions 604 are spongy material that conform to the manipulations of the thumb 104 and index finger 106 to effect good electrical contact between the conductive material of the first electrode 116 and the ears 208 by way of the spongy material being moved by the wearer of the gloves 102 to advance the contact to the ears 208. In some such embodiments, the extension 604 is a small piece of dry compressed sponge to which drops of water can be applied to cause acute expansion of the sponges during the scruffing procedure, causing extension of the electrical signal through the damp sponge to the ears 208.

In another embodiment, application of a conductive medium to fill the small region between the conductive material of the first electrode 116 on either or both of the index finger 106 and thumb 104 and the ears 208 causes extension of the electrical signal through the medium to the ears 208. The conductive medium could be: whipped cream, whipped cheese, whipped egg white, agar, gelatin, other organic electrically conductive mediums that hold, at least temporarily, an irregular shape and fill a small volume (<5 cubic cm).

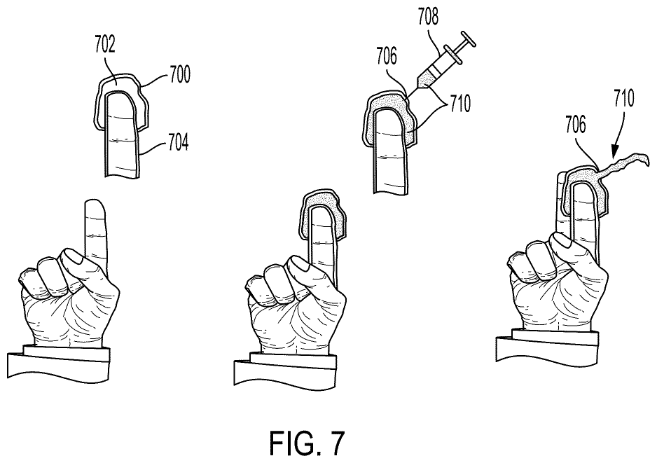

In another embodiment, vesicles containing the conductive medium can be configured into the fingertips, the squeezing action of the scruffing process causing the medium inside the vesicles to be expelled and fill the small volume between the conductive material and the ears. For example, in one embodiment, vesicles containing the conductive medium can be configured into or near the fingertips, the squeezing action of the scruffing process causing the medium inside the vesicles to escape and fill the small volume between the conductive material and the ears. In one such embodiment, a squeezable vessel is affixed to the dorsal side of the portion of the glove that extends over the index finger, depression of which can be accomplished by articulating an adjacent finger such as the middle finger, causing the contents of the vesicle to evacuate from the vesicle at the location of the index finger tip. In another embodiment seen in FIG. 7, a latex fingertip of the glove 102 is manufactured with an outer layer 700 at the index 106 and/or thumb 104 fingertips, extending about 0.25 to 0.75 inches from the tips and sealed at the circumference where the outer layer 700 terminates on the fingers. In this way, a small sealed space 702 is created between an inner layer 704 of the outer tip and the outer layer 700 of the inner tip. The material is such that a puncture 706 can be made with the point of a syringe 708 through which a medium 710 can be inserted and secured into the space 702. The puncture hole 706 is small enough to retain the medium 710, yet porous enough to allow the medium 710 to exit through the hole 706 when pressure is applied to the space 702.

In one embodiment there is a unique left-hand glove configured in a left-hand orientation and a unique right-hand glove configured in a right-hand orientation. In other embodiments, the pattern of the electrically conductive material is mirrored on the ventral and dorsal surfaces of the glove so that the glove can be worn on either hand and performed in an identical manner.

In one embodiment, the conductive material of one glove makes contact with an extremity of the animal, say the ears for example, and the conductive material of the other glove makes contact with another extremity of the animal, say tail or buttocks of an animal, such that the small voltage across the heart of the animals is detected and recorded.

In one embodiment, the glove superstructure itself is fabricated from a hearty material such as leather or cloth (nylon, polyester, acrylic, wool, etc.), with the conductive material strategically configured as described above, and is not designed to be readily disposable but re-usable.

In one embodiment, the gloves are infused or otherwise scented with lavender or other scents that are known or thought to reduce anxiety.

Taken together, the aforementioned glove for recording ECG in awake animals covers numerous scenarios, such as a mouse being scruffed, a small animal resting quietly or moving slowly atop or inside the gloved hand, a neonatal mouse, warmed by the palm and bridging, for example, the palm of the hand and the index finger, or an infant human, with one instrumented gloved hand supporting the neck of the child, and the other instrumented gloved hand supporting the child's buttocks.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may exist in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extent or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way that enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A biometric data collecting system for small animals, the system comprising:
a glove comprising thumb, index finger, middle finger, ring finger, little finger, and palm portions;
a first electrode comprising a conductive material disposed on the index finger portion and the thumb portion of the glove; and
a second electrode, electrically isolated from the first electrode, and comprising a conductive material disposed on the ring finger portion, little finger portion, and the palm portion below the ring finger portion and the little finger portion of the glove;
wherein, when a small animal is held by a wearer of the glove generally in the palm portion, an anterior of the animal is held between the index finger portion and thumb portion creating a first point of contact for the first electrode on the animal, and a posterior of the animal is held between the ring finger portion and/or little finger portion and the portion of the palm below the ring finger portion and little finger portion creating a second point of contact for the second electrode on the animal separate from the first point of contact; and
wherein biometric data is measured and collected using the first point of contact for the first electrode on the animal and the second point of contact for the second electrode on the animal separate from the first point of contact.

2. The system of claim 1, wherein the biometric data is electrocardiogram (EKG/ECG) data.

3. The system of claim 1, further comprising connectors that connect the first electrode and second electrode to biometric data collection devices.

4. The system of claim 3, wherein the connectors are magnetic connectors.

5. The system of claim 1, wherein the glove is configured in a left-hand orientation or a right-hand orientation.

6. The system of claim 1, wherein the electrically conductive material comprises a silver film, or a silver chloride film.

7. The system of claim 1, wherein the electrically conductive material comprises a conductive ink or paint.

8. The system of claim 1, wherein the glove comprises a latex or polymer glove.

9. The system of claim 1, wherein the glove comprises a leather or cloth glove.

10. The system of claim 1, further comprising:
a compressible material disposed between the palm of the glove and the conductive material of the second electrode on the portion of the palm below the ring and little finger.

11. The system of claim 1, further comprising:
a heating element disposed between the palm of the glove and the conductive material of the second electrode on the portion of the palm below the ring and little finger.

12. The system of claim 1, further comprising:
a force sensor disposed between the palm of the glove and the conductive material of the second electrode on the portion of the palm below the ring and little finger.

13. The system of claim 1, further comprising:
extensions of the first electrode disposed on the index finger and thumb configured to contact ears of the animal when the anterior of the animal is held between the index finger and thumb.

14. The system of claim 13, wherein the extensions comprise a compressible conductive material.

15. The system of claim 14, wherein the compressible conductive material comprises a sponge.

16. The system of claim 1, further comprising a conductive medium applied to the first electrode disposed on the index finger and thumb.

17. The system of claim 16, wherein the conductive medium is contained in vesicles disposed in fingertips of the glove where a pressure of holding the anterior of the animal causes the conductive medium to be expelled from the vesicles and applied to the first electrode disposed on the index finger and thumb.

18. The system of claim 17, wherein the conductive medium is expelled from the vesicles by pressure applied by an adjacent finger on the vesicle.

19. The system of claim 1, wherein the glove is scented.

20. The system of claim 1, further comprising:
a second glove comprising thumb, index finger, middle finger, ring finger, little finger, and palm portions;
a third electrode comprising a conductive material disposed on the index finger portion and thumb portion of the second glove; and
a fourth electrode independent from the third electrode comprising a conductive material disposed on the ring finger portion, little finger portion, and area of the palm portion below the ring finger portion and little finger portion of the second glove;
wherein the third electrode establishes a third point of contact and the fourth electrode establishes a fourth point of contact; and
wherein biometric data can be measured using combination of any of: the first point, second point, third point, and fourth point of contact on the small animal.

21. A method of using a biometric data collecting system for small animals, the method comprising:
providing a biometric data collecting system for small animals, the system comprising:
a glove comprising thumb, index finger, middle finger, ring finger, little finger, and palm portions;
a first electrode comprising a conductive material disposed on the index finger portion and the thumb portion of the glove; and
a second electrode, electrically isolated from the first electrode, and comprising a conductive material disposed on the ring finger portion, little finger portion, and the palm portion below the ring finger portion and the little finger portion of the glove;
wearing the biometric data collecting system on a hand of a user;
holding a small animal generally in the palm portion with an anterior of the animal held between the index finger portion and thumb portion creating a first point of contact for the first electrode on the animal, and a posterior of the animal held between the ring finger portion and/or little finger portion and the portion of the palm below the ring finger portion and little finger portion creating a second point of contact for the second electrode on the animal separate from the first point of contact; and
measuring and collecting biometric data using the first point of contact for the first electrode on the animal and the second point of contact for the second electrode on the animal separate from the first point of contact.

22. The method of claim 21 wherein the anterior of the animal comprises loose skin on a neck of the animal.

23. The method of claim 21 wherein the posterior of the animal comprises a tail of the animal.
